(19) United States
(12) Patent Application Publication
(10) Pub. No.: US 2005/0085759 A1
(43) Pub. Date: Apr. 21, 2005

(54) Pre-moistened swab apparatus

(76) Inventor: Dana L. Dwight, Fallbrook, CA (US)

Correspondence Address: S. Michael Bender, P.O. Box 530399, St. Petersburg, FL, 33747, US

(21) Appl. No.: 10/969,092

(22) Filed: Oct. 20, 2004

Related U.S. Application Data
(60) Provisional application No. 60/512,077, filed on Oct. 20, 2003.

(51) Int. Cl. A61M 35/00

(52) U.S. Cl. 604/2

(57) ABSTRACT

A pre-moistened swab apparatus includes a swab unit having a pair of swab portions located at opposite ends of a handle portion. One swab portion can be shaped round, and the other swab portion can be cone-shaped. Preferably, both swab portions are pre-moistened. A package is provided for containing swab units. The package includes a logo and other identifying indicia. Preferably, the package includes package walls which include transparent material. Preferably, the package includes a package opening which can used over and over again without being damaged. Preferably, the swab portions are pre-moistened with an aqueous solution which includes eye gentle and eye friendly ingredients. With the eye gentle and friendly ingredients employed in the swab portions, the user can wipe away makeup smudges with no residue left behind and can clean outer ear surfaces, sleep residues from eyes, pets eyes, etc.

### CROSS-REFERENCE TO RELATED APPLICATION

This application claims priority based upon my copending Provisional Application Ser. No. 60/512077, filed Oct. 20, 2003.

### BACKGROUND OF THE INVENTION

1. Field of the Invention

The present invention relates generally to swabs, and, more particularly, to swabs having an intermediate handle with separate swab portions affixed to opposite ends of the handle.

2. Description of the Prior Art

The use of swabs having a single swab affixed to one end of a handle is well known in the art. In addition the use of a swab having an intermediate handle with separate swab portions affixed to opposite ends of the handle is also well known in the art. In this respect, throughout the years, a number of innovations have been developed relating to swabs on the ends of handles, and the following U.S. patents are representative of some of those innovations: U.S. Pat. Nos. 4,863,422, 5,846,215, 5,947,986, 6,406,451, and 6,494,856.

More specifically, U.S. Pat. No. 4,863,422 discloses a double ended compressible tubular swab applicator comprising a length of tubular material containing a charge of a treating liquid in an innermost lumen and a heat generating material having microencapsulated water spheres embedded in an outermost lumen, for delivery of heated treating liquid upon compression of the microcapsules and rupturing of sealing members. For purposes of simplicity, it would be desirable if a swab device were provided in which two swab portions were provided that were pre-moistened without the need for heated treating liquid.

U.S. Pat. No. 5,846,215 discloses a swab which includes an elongate stem and an absorbent covering such as cotton surrounding opposite tips of the stem. An antimicrobial agent is dispersed within the absorbent covering. A process is also provided for manufacturing the swabs. The process includes the steps of preparing a liquid medium containing the antimicrobial agent, contacting the absorbent covering with the liquid medium and then drying the absorbent covering. The anti-bacterial agent is selected from the group consisting of halogenated salicylanilides, halogenated carbanilides, halogenated bisphenols, alkylbenzoylacrylates, quaternary ammonium compounds, thiuram sulfides, dithiocarbamates, antibiotics, halogenated diphenyl ethers, halogenated anilides of thiophene carboxylic acids, and chlorohexidines. Rather than having a swab portion having a material soaked therein and then dried, it would be desirable if a swab device were provided which have swab portions retained in a pre-moistened condition. In addition, there are other materials that may be desirable to provide in a pre-moistened swab portion. For example, it would be desirable if a swab device were pre-moistened with an aqueous solution containing Aloe; Barbadensis leaf juice; Rose, Sweet Violet and Elder Blossom water; witch hazel; cucumber; lemon; glycerin; carrageenan, and sucrose cocoate.

U.S. Pat. No. 5,947,986 discloses a hygienic applicator having a combination of moist and/or dry swab end sections made of absorbent material. The moist end section is pre-moistened with baby oil, cleaning solution, medicant, or any desired liquid. The dry end section can also be moistened with a preferred liquid and then dried, leaving this section impregnated with a desired residue. The end sections can be encapsulated with an easily removable wrap to prevent contamination. The wrap can be moisture-impermeable to retain the moisture of the wetted end sections. Wraps are incorporated for quickly unravelling the end sections just prior to use. As stated hereinabove, it would be desirable if a swab device were provided which has both swab portions retained in a pre-moistened condition.

U.S. Pat. No. 6,406,451 disclose an improved swab applicator which includes a swab connected to an elongate stick. The swab applicator further includes a sleeve encapsulating the swab and stick including a handle portion, receptacle portion and transition portion. The handle portion is preferably hollow and cylindrical in shape extending a substantial part around the length of the stick, while the receptacle portion of the sleeve is also hollow and cylindrically shaped having a substantially greater cross section than the handle portion. Meanwhile, the transition portion is preferably hollow and frusto conically shaped to connect the handle portion to the receptacle portion of the sleeve. The transition portion is substantially non-deformable when the receptacle portion of the sleeve is compressed creating a significant level of sheer stress to be imparted between the receptacle portion and transition portion of the sleeve causing the receptacle portion to sever from the rest of the sleeve. With this device, that packaging for the swab is severed when the package is opened, and the package cannot be used over and over again in a practical way. However, there may be times when it would be desirable to use a package for a swab over and over again. This would be especially true when multiple swabs are retained in a package, but the swabs are used only one at a time. In this respect, it would be desirable if a swab device were provided that was packaged in a package that can conveniently be used over and over again.

U.S. Pat. No. 6,494,856 discloses a swab product which includes a swab defined by an elongate stem and a fibrous absorbent covering such as cotton on at least one end of the stem. The fibrous absorbent covering is held together by a film-forming polymer. A treatment composition is dispersed onto the fibrous absorbent covering. Particularly preferred is a highly viscous hydrophobic treatment composition consisting of at least about 80% of petrolatum, which may serve to deliver actives such as bacitracin ointment or by itself serve as an occlusive therapeutic covering of skin to prevent infection.

In all of the patents mentioned above, the respective swab portions are round and bulbous shaped. For certain uses of a swab, a round and bulbous shape may not be desirable. For example, for cleaning a straight line with a round and bulbous shaped swab portion may be difficult to control on a straight line. Also, in eye corners, a round and bulbous shaped swab portion may not effectively reach the insides of the corners. In this respect, it would be desirable if a swab device were provided which includes a swab portion that has a pointed end. The pointed end would be easily controlled along a straight line and would be easy to reach into tight corners.

Thus, while the foregoing body of prior art indicates it to be well known to use swab devices, the prior art described above does not teach or suggest a pre-moistened swab apparatus which has the following combination of desirable features: (1) provides swabs that are pre-moistened without the need for heated treating liquid; (2) provides swab portions retained in a pre-moistened condition; (3) provides a package that can conveniently be used over and over again; (4) provides swab portions that are pre-moistened with an aqueous solution containing Aloe; Barbadensis leaf juice; Rose, Sweet Violet and Elder Blossom water; witch hazel; cucumber; lemon; glycerin; carrageenan, and sucrose cocoate; and (5) includes a swab portion that has a pointed end. The foregoing desired characteristics are provided by the unique pre-moistened swab apparatus of the present invention as will be made apparent from the following description thereof. Other advantages of the present invention over the prior art also will be rendered evident.

### SUMMARY OF THE INVENTION

To achieve the foregoing and other advantages, the present invention, briefly described, provides a pre-moistened swab apparatus which includes one or more swab units each of which includes a handle portion and at least one pre-moistened swab portion affixed to an end of a respective handle portion. A package is provided for containing the swab units. The package includes a logo and other identifying indicia.

Preferably, the package includes package walls which include transparent material. Preferably, the package includes a package opening which can used over and over again without being damaged.

The swab unit includes a pair of swab portions located at opposite ends of the handle portion. Preferably, one swab portion is cone-shaped, and the other swab portion is a rounded soft tip. The cone-shaped swab portion has a pointed end which can be used for more accurate cleaning of eye corners and straight lines. The swab portions are pre-moistened with an aqueous solution. Preferably, the aqueous solution includes eye gentle and friendly ingredients. With one preferred embodiment, the swab unit includes swab portions which are pre-moistened with an aqueous solution containing Aloe Vera gel, distilled water, witch hazel, and cucumber with a small amount of lemon. With another preferred embodiment, the swab unit includes swab portions which are pre-moistened with an aqueous solution containing Aloe; Barbadensis leaf juice; Rose, Sweet Violet and Elder Blossom water; witch hazel; cucumber; lemon; glycerin; carrageenan, and sucrose cocoate.

With the eye gentle and friendly ingredients employed in the swab portion, the user can wipe away makeup smudges, dirt, wax, and sleep residues with no residue left behind.

The above brief description sets forth rather broadly the more important features of the present invention in order that the detailed description thereof that follows may be better understood, and in order that the present contributions to the art may be better appreciated. There are, of course, additional features of the invention that will be described hereinafter and which will be for the subject matter of the claims appended hereto.

In this respect, before explaining at least two preferred embodiments of the invention in detail, it is understood that the invention is not limited in its application to the details of the construction and to the arrangements of the components set forth in the following description or illustrated in the drawings. The invention is capable of other embodiments and of being practiced and carried out in various ways. Also, it is to be understood, that the phraseology and terminology employed herein are for the purpose of description and should not be regarded as limiting.

As such, those skilled in the art will appreciate that the conception, upon which disclosure is based, may readily be utilized as a basis for designing other structures, methods, and systems for carrying out the several purposes of the present invention. It is important, therefore, that the claims be regarded as including such equivalent constructions insofar as they do not depart from the spirit and scope of the present invention.

It is therefore an object of the present invention to provide a new and improved pre-moistened swab apparatus which has all of the advantages of the prior art and none of the disadvantages.

It is another object of the present invention to provide a new and improved pre-moistened swab apparatus which may be easily and efficiently manufactured and marketed.

It is a further object of the present invention to provide a new and improved pre-moistened swab apparatus which is of durable and reliable construction.

An even further object of the present invention is to provide a new and improved pre-moistened swab apparatus which is susceptible of a low cost of manufacture with regard to both materials and labor, and which accordingly is then susceptible of low prices of sale to the consuming public, thereby making such pre-moistened swab apparatus available to the buying public.

Still yet a further object of the present invention is to provide a new and improved pre-moistened swab apparatus which provides swabs that are pre-moistened without the need for heated treating liquid.

Still another object of the present invention is to provide a new and improved pre-moistened swab apparatus that has swab portions retained in a pre-moistened condition.

Yet another object of the present invention is to provide a new and improved pre-moistened swab apparatus which includes a package that can conveniently be used over and over again.

Even another object of the present invention is to provide a new and improved pre-moistened swab apparatus that provides swab portions that are pre-moistened with an aqueous solution containing Aloe.

It is another object of the present invention to provide a new and improved pre-moistened swab apparatus which includes a swab portion that has a pointed end.

These together with still other objects of the invention, along with the various features of novelty which characterize the invention, are pointed out with particularity in the claims annexed to and forming a part of this disclosure. For a better understanding of the invention, its operating advantages and the specific objects attained by its uses, reference should be had to the accompanying drawings and descriptive matter in which there are illustrated preferred embodiments of the invention.

### BRIEF DESCRIPTION OF THE DRAWINGS

The invention will be better understood and the above objects as well as objects other than those set forth above will become more apparent after a study of the following detailed description thereof. Such description makes reference to the annexed drawing wherein:

FIG. 1 is a side view showing a first preferred embodiment of the pre-moistened swab apparatus of the invention contained in a package, and viewed from the front of the package.

FIG. 2 is an edge view of the embodiment of the pre-moistened swab apparatus shown in FIG. 1 taken along line 2-2 of FIG. 1.

FIG. 3 is a rear view of the embodiment of the pre-moistened swab apparatus of FIG. 1, viewed from the back of the package.

FIG. 4 is an enlarged partial cross-sectional view of an end portion of the handle portion and one pre-moistened swab portion.

FIG. 5 is a side view showing a second alternatively preferred embodiment of the pre-moistened swab apparatus of the invention contained in a package, and viewed from the front of the package.

FIG. 6 is an enlarged partial cross-sectional view of an end portion of the handle portion and one pre-moistened swab portion in the cone-shaped embodiment shown in FIG. 5.

### DESCRIPTION OF THE PREFERRED EMBODIMENT

With reference to the drawings, a new and improved pre-moistened swab apparatus embodying the principles and concepts of the present invention will be described.

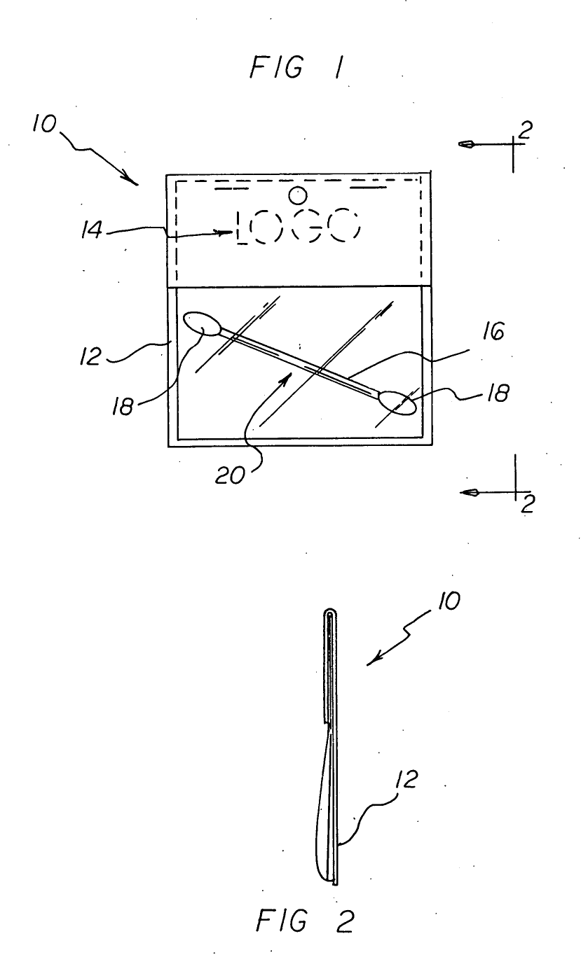

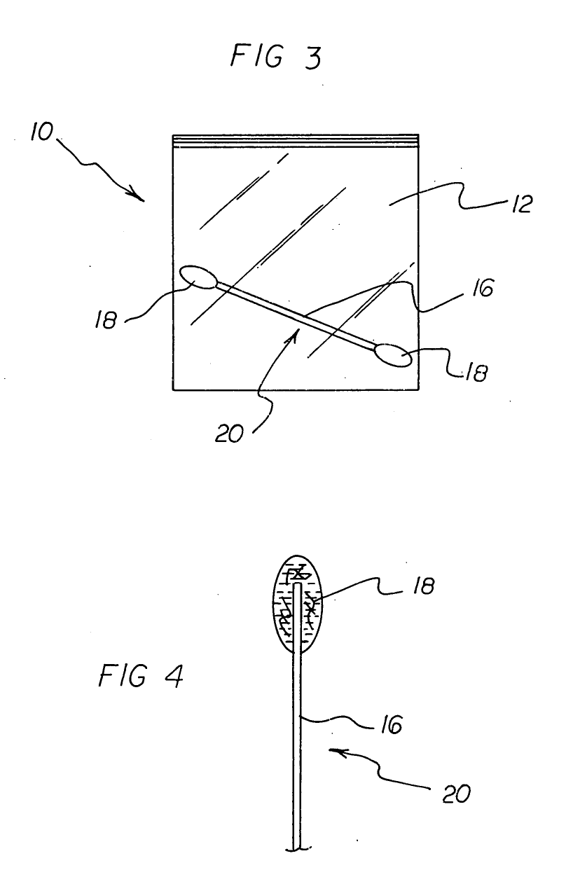

Turning to FIGS. 1-4, there is shown a first preferred embodiment of the pre-moistened swab apparatus of the invention generally designated by reference numeral 10. In the first preferred embodiment, pre-moistened swab apparatus 10 includes a pre-moistened swab apparatus 10 includes one or more swab units 20 each of which includes a handle portion 16 and at least one pre-moistened swab portion 18 affixed to an end of a respective handle portion 16. A package 12 is provided for containing the swab units 20. The package 12 includes a logo 14 and other identifying indicia.

Preferably, the package 12 includes package walls which include transparent material. Preferably, the package 12 includes a package opening which can used over and over again without being damaged.

The swab unit 20 includes a pair of swab portions 18 located at opposite ends of the handle portion 16. The swab unit 20 includes swab portions 18 which are pre-moistened with an aqueous solution. Preferably, the aqueous solution includes eye gentle and friendly ingredients. With a preferred embodiment, the swab unit 20 includes swab portions 18 which are pre-moistened with an aqueous solution containing Aloe Vera gel, distilled water, witch hazel, and cucumber with a small amount of lemon.

To use the pre-moistened swab apparatus 10 of the invention, a user grasps the pre-moistened swab apparatus 10 and opens the package 12 to remove the swab unit 20. Preferably, the package 12 can be opened and sealed numerous times without damaging the package 12. In this way, a single package 12 can be employed for carrying a plurality of swab units 20 that can be employed one at a time.

The user holds the swab unit 20 in a conventional way on the handle portion 16 to contact a swab portion 18 to a skin surface to remove smudges in makeup or to clean outer ear surfaces, sleep residues from eyes, pets eyes, etc.. With the eye gentle and friendly ingredients employed in the swab portion 18, the user can wipe away smudges with no residue left behind. The pre-moistened swab apparatus 10, with the one or more swab units 20 included in the package 12, can be designated “Make-up Mates”.

When the package 12 has package walls which includes transparent material, the user can take notice of how many unused swab units 20 remain in the package 12.

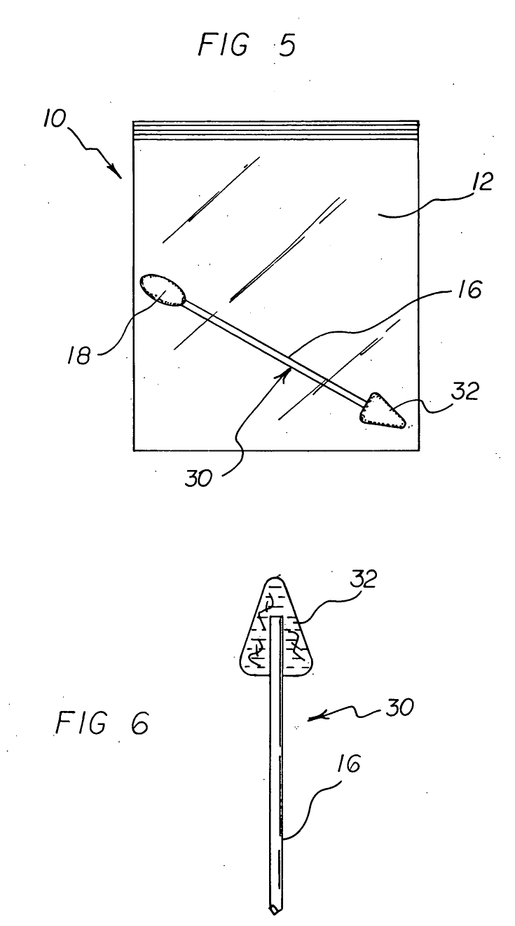

Turning FIGS. 5 and 6, a second alternatively preferred embodiment of the invention of the pre-moistened swab apparatus of the invention is generally designated by reference numeral 30. In the second preferred embodiment, the pre-moistened swab apparatus 30 includes one rounded, soft tip pre-moistened swab portion 18 connected to one end of an intermediate handle portion 16 and includes a cone-shaped swab portion 32 connected to the other end of the intermediate handle portion 16. The cone-shaped swab portion 32 has a pointed end which can be used for more accurate cleaning of eye corners and straight lines. A package 12 is provided for containing the swab units 30. The package 12 includes a logo 14 and other identifying indicia.

Preferably, the rounded, soft tip pre-moistened swab portion 18 and the cone-shaped swab portion 32 of the swab unit 30 are pre-moistened with an aqueous solution. Preferably, the aqueous solution includes eye gentle and friendly ingredients. In this respect, with another preferred embodiment, the swab unit 30 includes swab portions which are pre-moistened with an aqueous solution containing Aloe; Barbadensis leaf juice; Rose, Sweet Violet and Elder Blossom water; witch hazel; cucumber; lemon; glycerin; carrageenan, and sucrose cocoate.

The components of the pre-moistened swab apparatus of the invention can be made from inexpensive and durable cotton and plastic materials.

As to the manner of usage and operation of the instant invention, the same is apparent from the above disclosure, and accordingly, no further discussion relative to the manner of usage and operation need be provided.

It is apparent from the above that the present invention accomplishes all of the objects set forth by providing a new and improved pre-moistened swab apparatus that is low in cost, relatively simple in design and operation, and which advantageously provides swabs that are pre-moistened without the need for heated treating liquid. With the invention, a pre-moistened swab apparatus provides swab portions that retained in a pre-moistened condition. With the invention, a pre-moistened swab apparatus provides a package that can conveniently be used over and over again. With the invention, a pre-moistened swab apparatus includes a swab portion that has a pointed end permitting easy control along a straight line and in tight corners. With the invention, a pre-moistened swab apparatus provides swab portions that are pre-moistened with an aqueous solution containing Aloe; Barbadensis leaf juice; Rose, Sweet Violet and Elder Blossom water; witch hazel; cucumber; lemon; glycerin; carrageenan, and sucrose cocoate.

Thus, while the present invention has been shown in the drawings and fully described above with particularity and detail in connection with what is presently deemed to be the most practical and preferred embodiment(s) of the invention, it will be apparent to those of ordinary skill in the art that many modifications thereof may be made without departing from the principles and concepts set forth herein, including, but not limited to, variations in size, materials, shape, form, function and manner of operation, assembly and use.

Hence, the proper scope of the present invention should be determined only by the broadest interpretation of the appended claims so as to encompass all such modifications as well as all relationships equivalent to those illustrated in the drawings and described in the specification.

Finally, it will be appreciated that the purpose of the annexed Abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. Accordingly, the Abstract is neither intended to define the invention or the application, which only is measured by the claims, nor is it intended to be limiting as to the scope of the invention in any way.

1. A pre-moistened swab apparatus, comprising:
a swab unit which includes a handle portion and at least one pre-moistened swab portion which includes a pointed end affixed to an end of said handle portion.

2. The apparatus of claim 1 wherein said pointed end includes a cone-shaped swab portion.

3. The apparatus of claim 1 wherein said swab unit includes a pair of pre-moistened swab portions located at opposite ends of said handle portion.

4. The apparatus of claim 3 wherein said pair of pre-moistened swab portions includes a rounded, soft tip pre-moistened swab portion and a pre-moistened cone-shaped swab portion.

5. The apparatus of claim 1 wherein said swab unit includes a swab portion which is pre-moistened with an aqueous solution which includes eye gentle and eye friendly ingredients.

6. The apparatus of claim 5 wherein said swab unit includes a swab portion which is pre-moistened with an aqueous solution containing Aloe Vera gel, distilled water, witch hazel, and cucumber with a small amount of lemon.

7. The apparatus of claim 5 wherein said swab unit includes a swab portion which is pre-moistened with an aqueous solution containing Aloe; Barbadensis leaf juice; Rose, Sweet Violet and Elder Blossom water; witch hazel; cucumber; lemon; glycerin; carrageenan, and sucrose cocoate.

8. The apparatus of claim 1, further including:
a package for containing said swab unit.

9. The apparatus of claim 8 wherein said package includes a logo.

10. The apparatus of claim 8 wherein said package includes package walls which include transparent material.

11. The apparatus of claim 8 wherein said package includes a package opening which can be used over and over again without being damaged.

12. The apparatus of claim 1 wherein:
said swab unit includes a pair of pre-moistened swab portions located at opposite ends of said handle portion,
said pair of pre-moistened swab portions include a pre-moistened rounded, soft tip swab portion and a pre-moistened cone-shaped swab portion, and
said pair of pre-moistened swab portions are pre-moistened with an aqueous solution which includes eye gentle and eye friendly ingredients.

13. The apparatus of claim 12 wherein said swab unit includes a swab portion which is pre-moistened with an aqueous solution containing Aloe Vera gel, distilled water, witch hazel, and cucumber with a small amount of lemon.

14. The apparatus of claim 12 wherein said swab unit includes a swab portion which is pre-moistened with an aqueous solution containing Aloe; Barbadensis leaf juice; Rose, Sweet Violet and Elder Blossom water; witch hazel; cucumber; lemon; glycerin; carrageenan, and sucrose cocoate.

15. A pre-moistened swab apparatus, comprising:
a swab unit which includes a handle portion and at least one pre-moistened swab portion affixed to an end of said handle portion, and
wherein said pre-moistened swab portion is pre-moistened with an aqueous solution which includes eye gentle and eye friendly ingredients.

16. The apparatus of claim 15 wherein said pre-moistened swab portion includes an aqueous solution containing Aloe; Barbadensis leaf juice; Rose, Sweet Violet and Elder Blossom water; witch hazel; cucumber; lemon; glycerin; carrageenan, and sucrose cocoate.

17. The apparatus of claim 15 wherein said pre-moistened swab portion includes an aqueous solution containing Aloe Vera gel, distilled water, witch hazel, and cucumber with a small amount of lemon.
